(19) United States
(12) Patent Application Publication
(10) Pub. No.: US 2024/0307481 A1
(43) Pub. Date: Sep. 19, 2024

(54) COMPOSITIONS AND METHODS FOR IMPROVING EYE FUNCTION

(71) Applicant: William H. Cross, III, Waco, GA (US)

(72) Inventor: William H. Cross, III, Waco, GA (US)

(21) Appl. No.: 18/606,224

(22) Filed: Mar. 15, 2024

Related U.S. Application Data
(60) Provisional application No. 63/490,809, filed on Mar. 17, 2023.

(51) Int. Cl. A61K 38/05; A61K 31/01; A61K 31/047; A61K 31/09; A61K 31/145; A61K 31/198; A61K 31/51; A61K 38/06

(52) U.S. Cl. CPC A61K 38/05; A61K 31/01; A61K 31/047; A61K 31/09; A61K 31/145; A61K 31/198; A61K 31/51; A61K 38/063

(57) ABSTRACT

In accordance with the purpose(s) of the present disclosure, as embodied and broadly described herein, the disclosure, in one aspect, relates to unit dose compositions comprising (a) N-acetyl carnosine or a pharmaceutically acceptable salt thereof; (b) S-acetyl glutathione or a pharmaceutically acceptable salt thereof; (c) pterostilbene or a pharmaceutically acceptable salt thereof; (d) N-acetyl cysteine or a pharmaceutically acceptable salt thereof; (e) lutein or a pharmaceutically acceptable salt thereof; (f) taurine or a pharmaceutically acceptable salt thereof; (g) thiamine or a pharmaceutically acceptable salt thereof; (h) lycopene or a pharmaceutically acceptable salt thereof; and (i) zeaxanthin or a pharmaceutically acceptable salt thereof. In one aspect, the unit dose compositions improve eye function in a subject. In another aspect, the unit dose compositions treat or prevent eye disease is a subject.

### CROSS-REFERENCE TO RELATED APPLICATIONS

This application claims the benefit of and priority to co-pending U.S. Provisional Patent Application No. 63/490,809, filed on Mar. 17, 2023, the contents of which are incorporated by reference herein in their entireties.

### BACKGROUND

Diabetic eye disease is a leading cause of blindness and visual disability globally among adults. Initiation and progression of diabetic eye disease generally involves oxidative stress, apoptosis, mitochondrial dysfunction, and inflammation. For instance, increased oxidative stress of retinal cells damages the mitochondrial DNA in diabetic retinopathy, one of the most deleterious eye-related complications of diabetes. Oxidative stress is also a significant contributor to the pathophysiology of age-related cataract, a leading cause of blindness globally. There is currently a need for preventing or reducing eye disease and improving eye function subjects, particularly those that are diabetic or prediabetic.

### SUMMARY

In accordance with the purpose(s) of the present disclosure, as embodied and broadly described herein, the disclosure, in one aspect, relates to unit dose compositions comprising (a) N-acetyl carnosine or a pharmaceutically acceptable salt thereof; (b) S-acetyl glutathione or a pharmaceutically acceptable salt thereof; (c) pterostilbene or a pharmaceutically acceptable salt thereof; (d) N-acetyl cysteine or a pharmaceutically acceptable salt thereof; (e) lutein or a pharmaceutically acceptable salt thereof; (f) taurine or a pharmaceutically acceptable salt thereof; (g) thiamine or a pharmaceutically acceptable salt thereof; (h) lycopene or a pharmaceutically acceptable salt thereof; and (i) zeaxanthin or a pharmaceutically acceptable salt thereof. In one aspect, the unit dose compositions improve eye function in a subject. In another aspect, the unit dose compositions treat or prevent eye disease is a subject.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### DETAILED DESCRIPTION

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “an excipient” include, but are not limited to, mixtures or combinations of two or more such excipients, and the like.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1%to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5%to about 4.4%, and other possible sub-ranges) within the indicated range. Thus, for example, if a component is in an amount of about 1%, 2%, 3%, 4%, or 5%, where any value can be a lower and upper endpoint of a range, then any range is contemplated between 1% and 5% (e.g., 1% to 3%, 2% to 4%, etc.).

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, “administering” can refer to an administration that is oral, topical, intravenous, subcutaneous, transcutaneous, transdermal, intramuscular, intra-joint, parenteral, intra-arteriole, intradermal, intraventricular, intraosseous, intraocular, intracranial, intraperitoneal, intralesional, intranasal, intracardiac, intraarticular, intracavernous, intrathecal, intravireal, intracerebral, and intracerebroventricular, intratympanic, intracochlear, rectal, vaginal, by inhalation, by catheters, stents or via an implanted reservoir or other device that administers, either actively or passively (e.g. by diffusion) a composition the perivascular space and adventitia. For example, a medical device such as a stent can contain a composition or formulation disposed on its surface, which can then dissolve or be otherwise distributed to the surrounding tissue and cells. The term “parenteral” can include subcutaneous, intravenous, intramuscular, intra-articular, intra-synovial, intrasternal, intrathecal, intrahepatic, intralesional, and intracranial injections or infusion techniques. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used interchangeably herein, “subject,” “individual,” or “patient” can refer to a vertebrate organism, such as a mammal (e.g., human). “Subject” can also refer to a cell, a population of cells, a tissue, an organ, or an organism, preferably to human and constituents thereof.

As used herein, the terms “treating” and “treatment” can refer generally to obtaining a desired pharmacological and/or physiological effect. The effect can be, but does not necessarily have to be, prophylactic in terms of preventing or partially preventing a disease, symptom or condition thereof, such as, for example, eye disease. The effect can be therapeutic in terms of a partial or complete cure of a disease, condition, symptom or adverse effect attributed to the disease, disorder, or condition. The term “treatment” includes any one or more of the following: (a) preventing the disease from occurring in a subject which may be predisposed to the disease but has not yet been diagnosed as having it; (b) inhibiting the disease, i.e., arresting its development; and (c) relieving the disease, i.e., mitigating or ameliorating the disease and/or its symptoms or conditions. The term “treatment” as used herein can refer to both therapeutic treatment alone, prophylactic treatment alone, or both therapeutic and prophylactic treatment. Those in need of treatment (subjects in need thereof) can include those already with the disorder and/or those in which the disorder is to be prevented. As used herein, the term “treating”, can include inhibiting the disease, disorder or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected, e.g., such as treating the pain of a subject by administration of an analgesic agent even though such agent does not treat the cause of the pain.

As used herein, “therapeutic” can refer to treating, healing, and/or ameliorating a disease, disorder, condition, or side effect, or to decreasing in the rate of advancement of a disease, disorder, condition, or side effect.

As used herein, “effective amount” can refer to the amount of a disclosed compound or pharmaceutical composition provided herein that is sufficient to effect beneficial or desired biological, emotional, medical, or clinical response of a cell, tissue, system, animal, or human. An effective amount can be administered in one or more administrations, applications, or dosages. The term can also include within its scope amounts effective to enhance or restore to substantially normal physiological function.

For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. It is generally preferred that a maximum dose of the pharmacological agents of the invention (alone or in combination with other therapeutic agents) be used, that is, the highest safe dose according to sound medical judgment. It will be understood by those of ordinary skill in the art however, that a patient may insist upon a lower dose or tolerable dose for medical reasons, psychological reasons or for virtually any other reasons.

A response to a therapeutically effective dose of a disclosed compound and/or pharmaceutical composition, for example, can be measured by determining the physiological effects of the treatment or medication, such as the decrease or lack of disease symptoms following administration of the treatment or pharmacological agent. Other assays will be known to one of ordinary skill in the art and can be employed for measuring the level of the response. The amount of a treatment may be varied for example by increasing or decreasing the amount of a disclosed compound and/or pharmaceutical composition, by changing the disclosed compound and/or pharmaceutical composition administered, by changing the route of administration, by changing the dosage timing and so on. Dosage can vary, and can be administered in one or more dose administrations daily, for one or several days. Guidance can be found in the literature for appropriate dosages for given classes of pharmaceutical products.

As used herein, the term “prophylactically effective amount” refers to an amount effective for preventing onset or initiation of a disease or condition.

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, the term “improves,” “improvement,” or “improving” refers to enhancing one or more physical properties or features of a subject after being administered the unit dose compositions described herein. For example, prior to the administration of the unit dose composition, the subject may have a baseline glomerular filtration rate (GFR) of 40. After, the administration of the unit dose composition, the subject has a GFR of 60. Here, GFR of the subject improved 50%.

As used herein, the term “eye disease” refers to the gradual loss of eye function. Symptoms of eye disease include, but are not limited to, complete loss of vision, partial loss of vision, dry eye, or inflammation of any eye tissue or components.

As used herein, the term “eye function” refers to the ability of the eye to perform in its normal, healthy state.

The term “pharmaceutically acceptable” describes a material that is not biologically or otherwise undesirable, i.e., without causing an unacceptable level of undesirable biological effects or interacting in a deleterious manner.

The term “pharmaceutically acceptable salts”, as used herein, means salts of the active principal agents which are prepared with acids or bases that are tolerated by a biological system or tolerated by a subject or tolerated by a biological system and tolerated by a subject when administered in a therapeutically effective amount. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include, but are not limited to; sodium, potassium, calcium, ammonium, organic amino, magnesium salt, lithium salt, strontium salt or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include, but are not limited to; those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like.

As used herein, “dose,” “unit dose,” or “dosage” can refer to physically discrete units suitable for use in a subject, each unit containing a predetermined quantity of a disclosed compound and/or a pharmaceutical composition thereof calculated to produce the desired response or responses in association with its administration.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

Disclosed are the components to be used to prepare the compositions of the invention as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

It is understood that the compositions disclosed herein have certain functions. Disclosed herein are certain structural requirements for performing the disclosed functions, and it is understood that there are a variety of structures that can perform the same function that are related to the disclosed structures, and that these structures will typically achieve the same result.

As used herein, the terms “optional” or “optionally” means that the subsequently described event or circumstance can or cannot occur, and that the description includes instances where said event or circumstance occurs and instances where it does not.

Unless otherwise specified, temperatures referred to herein are based on atmospheric pressure (i.e., one atmosphere).

### Unit Dose Compositions and Methods of Making and Using the Same

In one aspect, disclosed herein are unit dose compositions for improving eye function in a subject. In another aspect, disclosed herein are unit dose compositions treating or preventing eye disease in a subject. In one aspect, the subject has diabetes or is prediabetic. Diabetes is increasingly recognized as the leading cause of eye failure. The unit dose compositions described herein can provide important health benefits to diabetics or prediabetics that are currently experiencing some degree of eye failure or disfunction. In the alternative, the unit dose compositions described herein can be taken prior to the onset of eye failure or disfunction as a prophylactic.

In one aspect, the unit dose composition reduces or prevents inflammation and oxidative stress on eye tissue. In another aspect, the unit dose composition reduces or prevents vision loss due to optic disc swelling, optic neuropathy, diabetic retinopathy, cataract. age-related macular degeneration (AMD), or glaucoma.

The unit dose compositions described herein can be administered to the subject on a daily basis for as long as needed to improve eye health and function of the subject. In one aspect, the subject is administered 1, 2, 3, 4, or 5 unit dose compositions per day. In one aspect, the unit dose composition is administered orally to the subject. In one aspect, the unit dose composition is in the form of a capsule, tablet, lozenge, or any other suitable form for oral administration.

In one aspect, the unit dose composition comprises (a) N-acetyl carnosine or a pharmaceutically acceptable salt thereof; (b) S-acetyl glutathione or a pharmaceutically acceptable salt thereof; (c) pterostilbene or a pharmaceutically acceptable salt thereof; (d) N-acetyl cysteine or a pharmaceutically acceptable salt thereof; (e) lutein or a pharmaceutically acceptable salt thereof; (f) taurine or a pharmaceutically acceptable salt thereof; (g) thiamine or a pharmaceutically acceptable salt thereof; (h) lycopene or a pharmaceutically acceptable salt thereof; and (i) zeaxanthin or a pharmaceutically acceptable salt thereof.

In one aspect, the amount of N-acetyl carnosine or the pharmaceutically acceptable salt thereof is from about 5 mg to about 100 mg per unit dose composition. In another aspect, the amount of N-acetyl carnosine or the pharmaceutically acceptable salt thereof is about 1 mg, 5 mg, 10 mg, 20 mg, 30 mg, 40 mg, 50 mg, 60 mg, 70 mg, 80 mg, 90 mg, or 100 mg per unit dose composition, where any value can be a lower and upper endpoint of a range (e.g., 10 mg to 40 mg). In one aspect, the pharmaceutically acceptable salt of N-acetyl carnosine is N-acetyl carnosine sulfate, N-acetyl carnosine chloride, or N-acetyl carnosine aspartate.

In one aspect, the amount of S-acetyl glutathione or the pharmaceutically acceptable salt thereof is from about 1 mg to about 100 mg per unit dose composition. In another aspect, the amount of S-acetyl glutathione or the pharmaceutically acceptable salt thereof is about 1 mg, 10 mg, 20 mg, 40 mg, 60 mg, 80 mg, 100 mg, 120 mg, 140 mg, 160 mg, 180 mg, or 200 mg per unit dose composition, where any value can be a lower and upper endpoint of a range (e.g., 20 mg to 80 mg). In one aspect, the pharmaceutically acceptable salt of S-acetyl glutathione is S-acetyl glutathione sulfate, S-acetyl glutathione chloride, or S-acetyl glutathione aspartate.

In one aspect, the amount of pterostilbene or the pharmaceutically acceptable salt thereof is from about 1 mg to about 200 mg per unit dose composition. In another aspect, the amount of pterostilbene or the pharmaceutically acceptable salt thereof is about 1 mg, 10 mg, 20 mg, 40 mg, 60 mg, 80 mg, 100 mg, 120 mg, 140 mg, 160 mg, 180 mg, or 200 mg per unit dose composition, where any value can be a lower and upper endpoint of a range (e.g., 20 mg to 80 mg). In one aspect, the pharmaceutically acceptable salt of pterostilbene is pterostilbene sulfate, pterostilbene chloride, or pterostilbene aspartate.

In one aspect, the amount of N-acetyl cysteine or the pharmaceutically acceptable salt thereof is from about 1 mg to about 200 mg per unit dose composition. In another aspect, the amount of N-acetyl cysteine or the pharmaceutically acceptable salt thereof is about 100 mg to about 1,000 mg, or about 100 mg, 200 mg, 300 mg, 400 mg, 500 mg, 600 mg, 700 mg, 800 mg, 900 mg, or 1,000 mg per unit dose composition, where any value can be a lower and upper endpoint of a range (e.g., 200 mg to 800 mg). In one aspect, the pharmaceutically acceptable salt of N-acetyl cysteine is N-acetyl cysteine sulfate, N-acetyl cysteine chloride, or N-acetyl cysteine aspartate.

In one aspect, the amount of lutein or the pharmaceutically acceptable salt thereof is from about 0.1 mg to about 50 mg per unit dose composition. In another aspect, the amount of lutein or the pharmaceutically acceptable salt thereof is about 0.1 mg, 1 mg, 5 mg, 10 mg, 15 mg, 20 mg, 25 mg, 30 mg, 35 mg, 40 mg, 45 mg, or 50 mg per unit dose composition, where any value can be a lower and upper endpoint of a range (e.g., 10 mg to 40 mg). In one aspect, the pharmaceutically acceptable salt of lutein is lutein sulfate, lutein chloride, or lutein aspartate.

In one aspect, the amount of taurine or the pharmaceutically acceptable salt thereof is from about 1 mg to about 200 mg per unit dose composition. In another aspect, the amount of taurine or the pharmaceutically acceptable salt thereof is about 100 mg to about 1,000 mg, or about 100 mg, 200 mg, 300 mg, 400 mg, 500 mg, 600 mg, 700 mg, 800 mg, 900 mg, or 1,000 mg per unit dose composition, where any value can be a lower and upper endpoint of a range (e.g., 200 mg to 800 mg). In one aspect, the pharmaceutically acceptable salt of taurine is taurine sulfate, taurine chloride, or taurine aspartate.

In one aspect, the amount of thiamine or the pharmaceutically acceptable salt thereof is from about 1 mg to about 200 mg per unit dose composition. In another aspect, the amount of thiamine or the pharmaceutically acceptable salt thereof is about 1 mg, 10 mg, 20 mg, 40 mg, 60 mg, 80 mg, 100 mg, 120 mg, 140 mg, 160 mg, 180 mg, or 200 mg per unit dose composition, where any value can be a lower and upper endpoint of a range (e.g., 20 mg to 80 mg). In one aspect, the pharmaceutically acceptable salt of thiamine is thiamine sulfate, thiamine chloride, or thiamine aspartate.

In one aspect, the amount of lycopene or the pharmaceutically acceptable salt thereof is from about 0.1 mg to about 50 mg per unit dose composition. In another aspect, the amount of lycopene or the pharmaceutically acceptable salt thereof is about 0.1 mg, 1 mg, 5 mg, 10 mg, 15 mg, 20 mg, 25 mg, 30 mg, 35 mg, 40 mg, 45 mg, or 50 mg per unit dose composition, where any value can be a lower and upper endpoint of a range (e.g., 10 mg to 40 mg). In one aspect, the pharmaceutically acceptable salt of lycopene is lycopene sulfate, lycopene chloride, or lycopene aspartate.

In one aspect, the amount of zeaxanthin or the pharmaceutically acceptable salt thereof is from about 0.1 mg to about 50 mg per unit dose composition. In another aspect, the amount of zeaxanthin or the pharmaceutically acceptable salt thereof is about 0.1 mg, 1 mg, 5 mg, 10 mg, 15 mg, 20 mg, 25 mg, 30 mg, 35 mg, 40 mg, 45 mg, or 50 mg per unit dose composition, where any value can be a lower and upper endpoint of a range (e.g., 10 mg to 40 mg). In one aspect, the pharmaceutically acceptable salt of zeaxanthin is zeaxanthin sulfate, zeaxanthin chloride, or zeaxanthin aspartate.

### Pharmaceutical Compositions

In various aspects, the unit dose compositions can include an optional pharmaceutically acceptable carrier to produce a pharmaceutical composition. As used herein, “pharmaceutically-acceptable carriers” means one or more of a pharmaceutically acceptable diluents, preservatives, antioxidants, solubilizers, emulsifiers, coloring agents, releasing agents, coating agents, sweetening, flavoring and perfuming agents, and adjuvants. The disclosed pharmaceutical compositions can be conveniently presented in unit dosage form and prepared by any of the methods well known in the art of pharmacy and pharmaceutical sciences.

In a further aspect, the disclosed pharmaceutical compositions comprise a therapeutically effective amount of at least one disclosed compound, at least one product of a disclosed method, or a pharmaceutically acceptable salt thereof as an active ingredient, a pharmaceutically acceptable carrier, optionally one or more other therapeutic agent, and optionally one or more adjuvant. The disclosed pharmaceutical compositions include those suitable for oral, rectal, topical, pulmonary, nasal, and parenteral administration, although the most suitable route in any given case will depend on the particular host and nature and severity of the conditions for which the active ingredient is being administered. In a further aspect, the disclosed pharmaceutical composition can be formulated to allow administration orally, nasally, via inhalation, parenterally, paracancerally, transmucosally, transdermally, intramuscularly, intravenously, intradermally, subcutaneously, intraperitoneally, intraventricularly, intracranially and intratumorally.

As used herein, “parenteral administration” includes administration by bolus injection or infusion, as well as administration by intravenous, intramuscular, intraarterial, intrathecal, intracapsular, intraorbital, intracardiac, intradermal, intraperitoneal, transtracheal, subcutaneous, subcuticular, intraarticular, subcapsular subarachnoid, intraspinal, epidural and intrasternal injection and infusion.

In various aspects, the present disclosure also relates to a pharmaceutical composition comprising a pharmaceutically acceptable carrier or diluent and, as active ingredient, a therapeutically effective amount of a disclosed compound, a product of a disclosed method of making, a pharmaceutically acceptable salt, a hydrate thereof, a solvate thereof, a polymorph thereof, or a stereochemically isomeric form thereof. In a further aspect, a disclosed compound, a product of a disclosed method of making, a pharmaceutically acceptable salt, a hydrate thereof, a solvate thereof, a polymorph thereof, or a stereochemically isomeric form thereof, or any subgroup or combination thereof may be formulated into various pharmaceutical forms for administration purposes.

In practice, the compounds of the present disclosure, or pharmaceutically acceptable salts thereof, of the present disclosure can be combined as the active ingredient in intimate admixture with a pharmaceutical carrier according to conventional pharmaceutical compounding techniques. The carrier can take a wide variety of forms depending on the form of preparation desired for administration, e.g., oral or parenteral (including intravenous). Thus, the pharmaceutical compositions of the present disclosure can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion or as a water-in-oil liquid emulsion. In addition to the common dosage forms set out above, the compounds of the present disclosure, and/or pharmaceutically acceptable salt(s) thereof, can also be administered by controlled release means and/or delivery devices. The compositions can be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

It is especially advantageous to formulate the aforementioned pharmaceutical compositions in unit dosage form for ease of administration and uniformity of dosage. The term “unit dosage form,” as used herein, refers to physically discrete units suitable as unitary dosages, each unit containing a predetermined quantity of active ingredient calculated to produce the desired therapeutic effect in association with the required pharmaceutical carrier. That is, a “unit dosage form” is taken to mean a single dose wherein all active and inactive ingredients are combined in a suitable system, such that the patient or person administering the drug to the patient can open a single container or package with the entire dose contained therein and does not have to mix any components together from two or more containers or packages. Typical examples of unit dosage forms are tablets (including scored or coated tablets), capsules or pills for oral administration; single dose vials for injectable solutions or suspension; suppositories for rectal administration; powder packets; wafers; and segregated multiples thereof. This list of unit dosage forms is not intended to be limiting in any way, but merely to represent typical examples of unit dosage forms.

The pharmaceutical compositions disclosed herein comprise a compound of the present disclosure (or pharmaceutically acceptable salts thereof) as an active ingredient, a pharmaceutically acceptable carrier, and optionally one or more additional therapeutic agents. In various aspects, the disclosed pharmaceutical compositions can include a pharmaceutically acceptable carrier and a disclosed compound, or a pharmaceutically acceptable salt thereof. In a further aspect, a disclosed compound, or pharmaceutically acceptable salt thereof, can also be included in a pharmaceutical composition in combination with one or more other therapeutically active compounds. The instant compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

Techniques and compositions for making dosage forms useful for materials and methods described herein are described, for example, in the following references: Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modern Pharmaceutics Drugs and the Pharmaceutical Sciences, Vol 40 (Gilbert S. Banker, Christopher T. Rhodes, Eds.).

In one aspect, the unit dose compositions described herein can be administered in admixture with suitable pharmaceutical diluents, excipients, extenders, or carriers (termed herein as a pharmaceutically acceptable carrier, or a carrier) suitably selected with respect to the intended form of administration and as consistent with conventional pharmaceutical practices. The deliverable compound will be in a form suitable for oral, rectal, topical, intravenous injection or parenteral administration. Carriers include solids or liquids, and the type of carrier is chosen based on the type of administration being used. The compounds may be administered as a dosage that has a known quantity of the compound.

Because of the ease in administration, oral administration can be a preferred dosage form, and tablets and capsules represent the most advantageous oral dosage unit forms in which case solid pharmaceutical carriers are obviously employed. However, other dosage forms may be suitable depending upon clinical population (e.g., age and severity of clinical condition), solubility properties of the specific disclosed compound used, and the like. Accordingly, the disclosed compounds can be used in oral dosage forms such as pills, powders, granules, elixirs, tinctures, suspensions, syrups, and emulsions. In preparing the compositions for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. Because of their ease of administration, tablets and capsules are the preferred oral dosage units whereby solid pharmaceutical carriers are employed. Optionally, tablets can be coated by standard aqueous or nonaqueous techniques.

The disclosed pharmaceutical compositions in an oral dosage form can comprise one or more pharmaceutical excipient and/or additive. Non-limiting examples of suitable excipients and additives include gelatin, natural sugars such as raw sugar or lactose, lecithin, pectin, starches (for example corn starch or amylose), dextran, polyvinyl pyrrolidone, polyvinyl acetate, gum arabic, alginic acid, tylose, talcum, lycopodium, silica gel (for example colloidal), cellulose, cellulose derivatives (for example cellulose ethers in which the cellulose hydroxy groups are partially etherified with lower saturated aliphatic alcohols and/or lower saturated, aliphatic oxyalcohols, for example methyl oxypropyl cellulose, methyl cellulose, hydroxypropyl methyl cellulose, hydroxypropyl methyl cellulose phthalate), fatty acids as well as magnesium, calcium or aluminum salts of fatty acids with 12 to 22 carbon atoms, in particular saturated (for example stearates), emulsifiers, oils and fats, in particular vegetable (for example, peanut oil, castor oil, olive oil, sesame oil, cottonseed oil, corn oil, wheat germ oil, sunflower seed oil, cod liver oil, in each case also optionally hydrated); glycerol esters and polyglycerol esters of saturated fatty acids C12H2402 to C18H3602 and their mixtures, it being possible for the glycerol hydroxy groups to be totally or also only partly esterified (for example mono-, di-and triglycerides); pharmaceutically acceptable mono-or multivalent alcohols and polyglycols such as polyethylene glycol and derivatives thereof, esters of aliphatic saturated or unsaturated fatty acids (2 to 22 carbon atoms, in particular 10-18 carbon atoms) with monovalent aliphatic alcohols (1 to 20 carbon atoms) or multivalent alcohols such as glycols, glycerol, diethylene glycol, pentacrythritol, sorbitol, mannitol and the like, which may optionally also be etherified, esters of citric acid with primary alcohols, acetic acid, urea, benzyl benzoate, dioxolanes, glyceroformals, tetrahydrofurfuryl alcohol, polyglycol ethers with C1-C12-alcohols, dimethylacetamide, lactamides, lactates, ethyl carbonates, silicones (in particular medium-viscous polydimethyl siloxanes), calcium carbonate, sodium carbonate, calcium phosphate, sodium phosphate, magnesium carbonate and the like.

Other auxiliary substances useful in preparing an oral dosage form are those which cause disintegration (so-called disintegrants), such as: cross-linked polyvinyl pyrrolidone, sodium carboxymethyl starch, sodium carboxymethyl cellulose or microcrystalline cellulose. Conventional coating substances may also be used to produce the oral dosage form. Those that may for example be considered are: polymerizates as well as copolymerizates of acrylic acid and/or methacrylic acid and/or their esters; copolymerizates of acrylic and methacrylic acid esters with a lower ammonium group content (for example EudragitR RS), copolymerizates of acrylic and methacrylic acid esters and trimethyl ammonium methacrylate (for example EudragitR RL); polyvinyl acetate; fats, oils, waxes, fatty alcohols; hydroxypropyl methyl cellulose phthalate or acetate succinate; cellulose acetate phthalate, starch acetate phthalate as well as polyvinyl acetate phthalate, carboxy methyl cellulose; methyl cellulose phthalate, methyl cellulose succinate,-phthalate succinate as well as methyl cellulose phthalic acid half ester; zein; ethyl cellulose as well as ethyl cellulose succinate; shellac, gluten; ethylcarboxyethyl cellulose; ethacrylate-maleic acid anhydride copolymer; maleic acid anhydride-vinyl methyl ether copolymer; styrol-maleic acid copolymerizate; 2-ethyl-hexyl-acrylate maleic acid anhydride; crotonic acid-vinyl acetate copolymer; glutaminic acid/glutamic acid ester copolymer; carboxymethylethylcellulose glycerol monooctanoate; cellulose acetate succinate; polyarginine.

Plasticizing agents that may be considered as coating substances in the disclosed oral dosage forms are: citric and tartaric acid esters (acetyl-triethyl citrate, acetyl tributyl-, tributyl-, triethyl-citrate); glycerol and glycerol esters (glycerol diacetate,-triacetate, acetylated monoglycerides, castor oil); phthalic acid esters (dibutyl-, diamyl-, diethyl-, dimethyl-, dipropyl-phthalate), di-(2-methoxy-or 2-ethoxyethyl)-phthalate, ethylphthalyl glycolate, butylphthalylethyl glycolate and butylglycolate; alcohols (propylene glycol, polyethylene glycol of various chain lengths), adipates (diethyladipate, di-(2-methoxy-or 2-ethoxyethyl)-adipate; benzophenone; diethyl-and diburylsebacate, dibutylsuccinate, dibutyltartrate; diethylene glycol dipropionate; ethyleneglycol diacetate, -dibutyrate, -dipropionate; tributyl phosphate, tributyrin; polyethylene glycol sorbitan monooleate (polysorbates such as Polysorbar 50); sorbitan monooleate.

Moreover, suitable binders, lubricants, disintegrating agents, coloring agents, flavoring agents, flow-inducing agents, and melting agents may be included as carriers. The pharmaceutical carrier employed can be, for example, a solid, liquid, or gas. Examples of solid carriers include, but are not limited to, lactose, terra alba, sucrose, glucose, methylcellulose, dicalcium phosphate, calcium sulfate, mannitol, sorbitol talc, starch, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In various aspects, a binder can include, for example, starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. In a further aspect, a disintegrator can include, for example, starch, methyl cellulose, agar, bentonite, xanthan gum, and the like.

In various aspects, an oral dosage form, such as a solid dosage form, can comprise a disclosed compound that is attached to polymers as targetable drug carriers or as a prodrug. Suitable biodegradable polymers useful in achieving controlled release of a drug include, for example, polylactic acid, polyglycolic acid, copolymers of polylactic and polyglycolic acid, caprolactones, polyhydroxy butyric acid, polyorthoesters, polyacetals, polydihydropyrans, polycyanoacylates, and hydrogels, preferably covalently crosslinked hydrogels.

Tablets may contain the active ingredient in admixture with non-toxic pharmaceutically acceptable excipients which are suitable for the manufacture of tablets. These excipients may be, for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period.

A tablet containing a disclosed compound can be prepared by compression or molding, optionally with one or more accessory ingredients or adjuvants. Compressed tablets can be prepared by compressing, in a suitable machine, the active ingredient in a free-flowing form such as powder or granules, optionally mixed with a binder, lubricant, inert diluent, surface active or dispersing agent. Molded tablets can be made by molding in a suitable machine, a mixture of the powdered compound moistened with an inert liquid diluent.

In various aspects, a solid oral dosage form, such as a tablet, can be coated with an enteric coating to prevent ready decomposition in the stomach. In various aspects, enteric coating agents include, but are not limited to, hydroxypropylmethylcellulose phthalate, methacrylic acid-methacrylic acid ester copolymer, polyvinyl acetate-phthalate and cellulose acetate phthalate. Akihiko Hasegawa “Application of solid dispersions of Nifedipine with enteric coating agent to prepare a sustained-release dosage form” Chem. Pharm. Bull. 33:1615-1619 (1985). Various enteric coating materials may be selected on the basis of testing to achieve an enteric coated dosage form designed ab initio to have a preferable combination of dissolution time, coating thicknesses and diametral crushing strength (e.g., see S. C. Porter et al. “The Properties of Enteric Tablet Coatings Made From Polyvinyl Acetate-phthalate and Cellulose acetate Phthalate”, J. Pharm. Pharmacol. 22:42p (1970)). In a further aspect, the enteric coating may comprise hydroxypropyl-methylcellulose phthalate, methacrylic acid-methacrylic acid ester copolymer, polyvinyl acetate-phthalate and cellulose acetate phthalate.

In various aspects, an oral dosage form can be a solid dispersion with a water soluble or a water insoluble carrier. Examples of water soluble or water insoluble carrier include, but are not limited to, polyethylene glycol, polyvinylpyrrolidone, hydroxypropylmethyl-cellulose, phosphatidylcholine, polyoxyethylene hydrogenated castor oil, hydroxypropylmethylcellulose phthalate, carboxymethylethylcellulose, or hydroxypropylmethylcellulose, ethyl cellulose, or stearic acid.

In various aspects, an oral dosage form can be in a liquid dosage form, including those that are ingested, or alternatively, administered as a mouth wash or gargle. For example, a liquid dosage form can include aqueous suspensions, which contain the active materials in admixture with excipients suitable for the manufacture of aqueous suspensions. In addition, oily suspensions may be formulated by suspending the active ingredient in a vegetable oil, for example arachis oil, olive oil, sesame oil or coconut oil, or in a mineral oil such as liquid paraffin. Oily suspensions may also contain various excipients. The pharmaceutical compositions of the present disclosure may also be in the form of oil-in-water emulsions, which may also contain excipients such as sweetening and flavoring agents.

For the preparation of solutions or suspensions it is, for example, possible to use water, particularly sterile water, or physiologically acceptable organic solvents, such as alcohols (ethanol, propanol, isopropanol, 1,2-propylene glycol, polyglycols and their derivatives, fatty alcohols, partial esters of glycerol), oils (for example peanut oil, olive oil, sesame oil, almond oil, sunflower oil, soya bean oil, castor oil, bovine hoof oil), paraffins, dimethyl sulfoxide, triglycerides and the like.

In the case of a liquid dosage form such as a drinkable solutions, the following substances may be used as stabilizers or solubilizers: lower aliphatic mono-and multivalent alcohols with 2-4 carbon atoms, such as ethanol, n-propanol, glycerol, polyethylene glycols with molecular weights between 200-600 (for example 1 to 40% aqueous solution), diethylene glycol monoethyl ether, 1,2-propylene glycol, organic amides, for example amides of aliphatic C1-C6-carboxylic acids with ammonia or primary, secondary or tertiary C1-C4-amines or C1-C4-hydroxy amines such as urea, urethane, acetamide, N-methyl acetamide, N,N-diethyl acetamide, N,N-dimethyl acetamide, lower aliphatic amines and diamines with 2-6 carbon atoms, such as ethylene diamine, hydroxyethyl theophylline, tromethamine (for example as 0.1 to 20% aqueous solution), aliphatic amino acids.

In preparing the disclosed liquid dosage form can comprise solubilizers and emulsifiers such as the following non-limiting examples can be used: polyvinyl pyrrolidone, sorbitan fatty acid esters such as sorbitan trioleate, phosphatides such as lecithin, acacia, tragacanth, polyoxyethylated sorbitan monooleate and other ethoxylated fatty acid esters of sorbitan, polyoxyethylated fats, polyoxyethylated oleotriglycerides, linolizated oleotriglycerides, polyethylene oxide condensation products of fatty alcohols, alkylphenols or fatty acids or also 1-methyl-3-(2-hydroxyethyl)imidazolidone-(2). In this context, polyoxyethylated means that the substances in question contain polyoxyethylene chains, the degree of polymerization of which generally lies between 2 and 40 and in particular between 10 and 20. Polyoxyethylated substances of this kind may for example be obtained by reaction of hydroxyl group-containing compounds (for example mono-or diglycerides or unsaturated compounds such as those containing oleic acid radicals) with ethylene oxide (for example 40 Mol ethylene oxide per 1 Mol glyceride). Examples of oleotriglycerides are olive oil, peanut oil, castor oil, sesame oil, cottonseed oil, corn oil. See also Dr. H. P. Fiedler “Lexikon der Hillsstoffe für Pharmazie, Kostnetik und angrenzende Gebiete” 1971, pages 191-195.

In various aspects, a liquid dosage form can further comprise preservatives, stabilizers, buffer substances, flavor correcting agents, sweeteners, colorants, antioxidants and complex formers and the like. Complex formers which may be for example be considered are chelate formers such as ethylene diamine retrascetic acid, nitrilotriacetic acid, diethylene triamine pentacetic acid and their salts.

It may optionally be necessary to stabilize a liquid dosage form with physiologically acceptable bases or buffers to a pH range of approximately 6 to 9. Preference may be given to as neutral or weakly basic a pH value as possible (up to pH 8).

In order to enhance the solubility and/or the stability of a disclosed compound in a disclosed liquid dosage form, a parenteral injection form, or an intravenous injectable form, it can be advantageous to employ α-, β-or γ-cyclodextrins or their derivatives, in particular hydroxyalkyl substituted cyclodextrins, e.g., 2-hydroxypropyl-β-cyclodextrin or sulfobutyl-β-cyclodextrin. Also, co-solvents such as alcohols may improve the solubility and/or the stability of the compounds according to the present disclosure in pharmaceutical compositions.

In various aspects, a disclosed liquid dosage form, a parenteral injection form, or an intravenous injectable form can further comprise liposome delivery systems, such as small unilamellar vesicles, large unilamellar vesicles, and multilamellar vesicles. Liposomes can be formed from a variety of phospholipids, such as cholesterol, stearylamine, or phosphatidylcholines.

Pharmaceutical compositions of the present disclosure suitable injection, such as parenteral administration, such as intravenous, intramuscular, or subcutaneous administration. Pharmaceutical compositions for injection can be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present disclosure suitable for parenteral administration can include sterile aqueous or oleaginous solutions, suspensions, or dispersions. Furthermore, the compositions can be in the form of sterile powders for the extemporaneous preparation of such sterile injectable solutions or dispersions. In some aspects, the final injectable form is sterile and must be effectively fluid for use in a syringe. The pharmaceutical compositions should be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Injectable solutions, for example, can be prepared in which the carrier comprises saline solution, glucose solution or a mixture of saline and glucose solution. Injectable suspensions may also be prepared in which case appropriate liquid carriers, suspending agents and the like may be employed. In some aspects, a disclosed parenteral formulation can comprise about 0.01-0.1 M, e.g. about 0.05 M, phosphate buffer. In a further aspect, a disclosed parenteral formulation can comprise about 0.9% saline.

In various aspects, a disclosed parenteral pharmaceutical composition can comprise pharmaceutically acceptable carriers such as aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include but not limited to water, alcoholic/aqueous solutions, emulsions or suspensions, including saline and buffered media. Parenteral vehicles can include mannitol, normal serum albumin, sodium chloride solution, Ringer's dextrose, dextrose and sodium chloride, lactated Ringer's and fixed oils. Intravenous vehicles include fluid and nutrient replenishers, electrolyte replenishers such as those based on Ringer's dextrose, and the like. Preservatives and other additives may also be present, such as, for example, antimicrobials, antioxidants, collating agents, inert gases and the like. In a further aspect, a disclosed parenteral pharmaceutical composition can comprise may contain minor amounts of additives such as substances that enhance isotonicity and chemical stability, e.g., buffers and preservatives. Also contemplated for injectable pharmaceutical compositions are solid form preparations that are intended to be converted, shortly before use, to liquid form preparations. Furthermore, other adjuvants can be included to render the formulation isotonic with the blood of the subject or patient.

In addition to the pharmaceutical compositions described herein above, the disclosed compounds can also be formulated as a depot preparation. Such long-acting formulations can be administered by implantation (e.g., subcutaneously or intramuscularly) or by intramuscular injection. Thus, for example, the compounds can be formulated with suitable polymeric or hydrophobic materials (e.g., as an emulsion in an acceptable oil) or ion exchange resins, or as sparingly soluble derivatives, e.g., as a sparingly soluble salt.

Pharmaceutical compositions of the present disclosure can be in a form suitable for topical administration. As used herein, the phrase “topical application” means administration onto a biological surface, whereby the biological surface includes, for example, a skin area (e.g., hands, forearms, elbows, legs, face, nails, anus and genital areas) or a mucosal membrane. By selecting the appropriate carrier and optionally other ingredients that can be included in the composition, as is detailed herein below, the compositions of the present invention may be formulated into any form typically employed for topical application. A topical pharmaceutical composition can be in a form of a cream, an ointment, a paste, a gel, a lotion, milk, a suspension, an aerosol, a spray, foam, a dusting powder, a pad, and a patch. Further, the compositions can be in a form suitable for use in transdermal devices. These formulations can be prepared, utilizing a compound of the present disclosure, or pharmaceutically acceptable salts thereof, via conventional processing methods. As an example, a cream or ointment is prepared by mixing hydrophilic material and water, together with about 5 wt % to about 10 wt % of the compound, to produce a cream or ointment having a desired consistency.

In the compositions suitable for percutaneous administration, the carrier optionally comprises a penetration enhancing agent and/or a suitable wetting agent, optionally combined with suitable additives of any nature in minor proportions, which additives do not introduce a significant deleterious effect on the skin. Said additives may facilitate the administration to the skin and/or may be helpful for preparing the desired compositions. These compositions may be administered in various ways, e.g., as a transdermal patch, as a spot-on, as an ointment.

Ointments are semisolid preparations, typically based on petrolatum or petroleum derivatives. The specific ointment base to be used is one that provides for optimum delivery for the active agent chosen for a given formulation, and, preferably, provides for other desired characteristics as well (e.g., emollience). As with other carriers or vehicles, an ointment base should be inert, stable, nonirritating and nonsensitizing. As explained in Remington: The Science and Practice of Pharmacy, 19th Ed., Easton, Pa.: Mack Publishing Co. (1995), pp. 1399-1404, ointment bases may be grouped in four classes: oleaginous bases; emulsifiable bases; emulsion bases; and water-soluble bases. Oleaginous ointment bases include, for example, vegetable oils, fats obtained from animals, and semisolid hydrocarbons obtained from petroleum. Emulsifiable ointment bases, also known as absorbent ointment bases, contain little or no water and include, for example, hydroxystearin sulfate, anhydrous lanolin and hydrophilic petrolatum. Emulsion ointment bases are either water-in-oil (W/O) emulsions or oil-in-water (O/W) emulsions, and include, for example, cetyl alcohol, glyceryl monostearate, lanolin and stearic acid. Preferred water-soluble ointment bases are prepared from polyethylene glycols of varying molecular weight.

Lotions are preparations that are to be applied to the skin surface without friction. Lotions are typically liquid or semiliquid preparations in which solid particles, including the active agent, are present in a water or alcohol base. Lotions are typically preferred for treating large body areas, due to the ease of applying a more fluid composition. Lotions are typically suspensions of solids, and oftentimes comprise a liquid oily emulsion of the oil-in-water type. It is generally necessary that the insoluble matter in a lotion be finely divided. Lotions typically contain suspending agents to produce better dispersions as well as compounds useful for localizing and holding the active agent in contact with the skin, such as methylcellulose, sodium carboxymethyl-cellulose, and the like.

Creams are viscous liquids or semisolid emulsions, either oil-in-water or water-in-oil. Cream bases are typically water-washable, and contain an oil phase, an emulsifier and an aqueous phase. The oil phase, also called the “internal” phase, is generally comprised of petrolatum and/or a fatty alcohol such as cetyl or stearyl alcohol. The aqueous phase typically, although not necessarily, exceeds the oil phase in volume, and generally contains a humectant. The emulsifier in a cream formulation is generally a nonionic, anionic, cationic or amphoteric surfactant. Reference may be made to Remington: The Science and Practice of Pharmacy, supra, for further information.

Pastes are semisolid dosage forms in which the bioactive agent is suspended in a suitable base. Depending on the nature of the base, pastes are divided between fatty pastes or those made from a single-phase aqueous gel. The base in a fatty paste is generally petrolatum, hydrophilic petrolatum and the like. The pastes made from single-phase aqueous gels generally incorporate carboxymethylcellulose or the like as a base. Additional reference may be made to Remington: The Science and Practice of Pharmacy, for further information.

Gel formulations are semisolid, suspension-type systems. Single-phase gels contain organic macromolecules distributed substantially uniformly throughout the carrier liquid, which is typically aqueous, but also, preferably, contain an alcohol and, optionally, an oil. Preferred organic macromolecules, i.e., gelling agents, are crosslinked acrylic acid polymers such as the family of carbomer polymers, e.g., carboxypolyalkylenes that may be obtained commercially under the trademark Carbopol™. Other types of preferred polymers in this context are hydrophilic polymers such as polyethylene oxides, polyoxyethylene-polyoxypropylene copolymers and polyvinylalcohol; modified cellulose, such as hydroxypropyl cellulose, hydroxyethyl cellulose, hydroxypropyl methylcellulose, hydroxypropyl methylcellulose phthalate, and methyl cellulose; gums such as tragacanth and xanthan gum; sodium alginate; and gelatin. In order to prepare a uniform gel, dispersing agents such as alcohol or glycerin can be added, or the gelling agent can be dispersed by trituration, mechanical mixing or stirring, or combinations thereof.

Sprays generally provide the active agent in an aqueous and/or alcoholic solution which can be misted onto the skin for delivery. Such sprays include those formulated to provide for concentration of the active agent solution at the site of administration following delivery, e.g., the spray solution can be primarily composed of alcohol or other like volatile liquid in which the active agent can be dissolved. Upon delivery to the skin, the carrier evaporates, leaving concentrated active agent at the site of administration.

Foam compositions are typically formulated in a single or multiple phase liquid form and housed in a suitable container, optionally together with a propellant which facilitates the expulsion of the composition from the container, thus transforming it into a foam upon application. Other foam forming techniques include, for example the “Bag-in-a-can” formulation technique. Compositions thus formulated typically contain a low-boiling hydrocarbon, e.g., isopropane. Application and agitation of such a composition at the body temperature cause the isopropane to vaporize and generate the foam, in a manner similar to a pressurized aerosol foaming system. Foams can be water-based or aqueous alkanolic, but are typically formulated with high alcohol content which, upon application to the skin of a user, quickly evaporates, driving the active ingredient through the upper skin layers to the site of treatment.

Skin patches typically comprise a backing, to which a reservoir containing the active agent is attached. The reservoir can be, for example, a pad in which the active agent or composition is dispersed or soaked, or a liquid reservoir. Patches typically further include a frontal water permeable adhesive, which adheres and secures the device to the treated region. Silicone rubbers with self-adhesiveness can alternatively be used. In both cases, a protective permeable layer can be used to protect the adhesive side of the patch prior to its use. Skin patches may further comprise a removable cover, which serves for protecting it upon storage.

Examples of patch configuration which can be utilized with the present invention include a single-layer or multi-layer drug-in-adhesive systems which are characterized by the inclusion of the drug directly within the skin-contacting adhesive. In such a transdermal patch design, the adhesive not only serves to affix the patch to the skin, but also serves as the formulation foundation, containing the drug and all the excipients under a single backing film. In the multi-layer drug-in-adhesive patch a membrane is disposed between two distinct drug-in-adhesive layers or multiple drug-in-adhesive layers are incorporated under a single backing film.

Examples of pharmaceutically acceptable carriers that are suitable for pharmaceutical compositions for topical applications include carrier materials that are well-known for use in the cosmetic and medical arts as bases for e.g., emulsions, creams, aqueous solutions, oils, ointments, pastes, gels, lotions, milks, foams, suspensions, aerosols and the like, depending on the final form of the composition. Representative examples of suitable carriers according to the present invention therefore include, without limitation, water, liquid alcohols, liquid glycols, liquid polyalkylene glycols, liquid esters, liquid amides, liquid protein hydrolysates, liquid alkylated protein hydrolysates, liquid lanolin and lanolin derivatives, and like materials commonly employed in cosmetic and medicinal compositions. Other suitable carriers according to the present invention include, without limitation, alcohols, such as, for example, monohydric and polyhydric alcohols, e.g., ethanol, isopropanol, glycerol, sorbitol, 2-methoxyethanol, diethyleneglycol, ethylene glycol, hexyleneglycol, mannitol, and propylene glycol; ethers such as diethyl or dipropyl ether; polyethylene glycols and methoxypolyoxyethylenes (carbowaxes having molecular weight ranging from 200 to 20,000); polyoxyethylene glycerols, polyoxyethylene sorbitols, stearoyl diacetin, and the like.

Topical compositions of the present disclosure can, if desired, be presented in a pack or dispenser device, such as an FDA-approved kit, which may contain one or more unit dosage forms containing the active ingredient. The dispenser device may, for example, comprise a tube. The pack or dispenser device may be accompanied by instructions for administration. The pack or dispenser device may also be accompanied by a notice in a form prescribed by a governmental agency regulating the manufacture, use, or sale of pharmaceuticals, which notice is reflective of approval by the agency of the form of the compositions for human or veterinary administration. Such notice, for example, may include labeling approved by the U.S. Food and Drug Administration for prescription drugs or of an approved product insert. Compositions comprising the topical composition of the invention formulated in a pharmaceutically acceptable carrier may also be prepared, placed in an appropriate container, and labeled for treatment of an indicated condition.

Another patch system configuration which can be used by the present invention is a reservoir transdermal system design which is characterized by the inclusion of a liquid compartment containing a drug solution or suspension separated from the release liner by a semi-permeable membrane and adhesive. The adhesive component of this patch system can either be incorporated as a continuous layer between the membrane and the release liner or in a concentric configuration around the membrane. Yet another patch system configuration which can be utilized by the present invention is a matrix system design which is characterized by the inclusion of a semisolid matrix containing a drug solution or suspension which is in direct contact with the release liner. The component responsible for skin adhesion is incorporated in an overlay and forms a concentric configuration around the semisolid matrix.

Pharmaceutical compositions of the present disclosure can be in a form suitable for rectal administration wherein the carrier is a solid. It is preferable that the mixture forms unit dose suppositories. Suitable carriers include cocoa butter and other materials commonly used in the art. The suppositories can be conveniently formed by first admixing the composition with the softened or melted carrier(s) followed by chilling and shaping in molds.

Pharmaceutical compositions containing a compound of the present disclosure, and/or pharmaceutically acceptable salts thereof, can also be prepared in powder or liquid concentrate form.

The pharmaceutical composition (or formulation) may be packaged in a variety of ways. Generally, an article for distribution includes a container that contains the pharmaceutical composition in an appropriate form. Suitable containers are well known to those skilled in the art and include materials such as bottles (plastic and glass), sachets, foil blister packs, and the like. The container may also include a tamper proof assemblage to prevent indiscreet access to the contents of the package. In addition, the container typically has deposited thereon a label that describes the contents of the container and any appropriate warnings or instructions.

The disclosed pharmaceutical compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Pharmaceutical compositions comprising a disclosed compound formulated in a compatible pharmaceutical carrier may also be prepared, placed in an appropriate container, and labeled for treatment of an indicated condition.

The exact dosage and frequency of administration depends on the particular disclosed compound, a product of a disclosed method of making, a pharmaceutically acceptable salt, solvate, or polymorph thereof, a hydrate thereof, a solvate thereof, a polymorph thereof, or a stereochemically isomeric form thereof; the particular condition being treated and the severity of the condition being treated; various factors specific to the medical history of the subject to whom the dosage is administered such as the age; weight, sex, extent of disorder and general physical condition of the particular subject, as well as other medication the individual may be taking; as is well known to those skilled in the art. Furthermore, it is evident that said effective daily amount may be lowered or increased depending on the response of the treated subject and/or depending on the evaluation of the physician prescribing the compounds of the present disclosure.

Depending on the mode of administration, the pharmaceutical composition will comprise from 0.05 to 99% by weight, preferably from 0.1 to 70% by weight, more preferably from 0.1 to 50% by weight of the active ingredients or components, and, from 1 to 99.95% by weight, preferably from 30 to 99.9% by weight, more preferably from 50 to 99.9% by weight of a pharmaceutically acceptable carrier, all percentages being based on the total weight of the composition.

The disclosed pharmaceutical compositions can further comprise other therapeutically active compounds, which are usually applied in the treatment of the above mentioned pathological or clinical conditions.

It is understood that the disclosed compositions can be prepared from the disclosed compounds. It is also understood that the disclosed compositions can be employed in the disclosed methods of using.

As already mentioned, the present disclosure relates to a pharmaceutical composition comprising a therapeutically effective amount of a disclosed compound, a product of a disclosed method of making, a pharmaceutically acceptable salt, a hydrate thereof, a solvate thereof, a polymorph thereof, and a pharmaceutically acceptable carrier. Additionally, the present disclosure relates to a process for preparing such a pharmaceutical composition, characterized in that a pharmaceutically acceptable carrier is intimately mixed with a therapeutically effective amount of a compound according to the present disclosure.

### Aspects

Aspect 1. A unit dose composition comprising
(a) N-acetyl carnosine or a pharmaceutically acceptable salt thereof;
(b) S-acetyl glutathione or a pharmaceutically acceptable salt thereof;
(c) pterostilbene or a pharmaceutically acceptable salt thereof;
(d) N-acetyl cysteine or a pharmaceutically acceptable salt thereof;
(e) lutein or a pharmaceutically acceptable salt thereof;
(f) taurine or a pharmaceutically acceptable salt thereof;
(g) thiamine or a pharmaceutically acceptable salt thereof;
(h) lycopene or a pharmaceutically acceptable salt thereof; and
(i) zeaxanthin or a pharmaceutically acceptable salt thereof.

Aspect 2. The unit dose composition of Aspect 1, wherein N-acetyl carnosine or the pharmaceutically acceptable salt thereof is from about 1 mg to about 100 mg per unit dose composition, about 5 mg to about 50 mg per unit dose composition, or about 10 mg to about 35 mg per unit dose composition.

Aspect 3. The unit dose composition of Aspect 1, wherein S-acetyl glutathione or the pharmaceutically acceptable salt thereof is from about 1 mg to about 200 mg per unit dose composition, about 5 mg to about 100 mg per unit dose composition, or about 25 mg to about 75 mg per unit dose composition.

Aspect 4. The unit dose composition of Aspect 1, wherein pterostilbene or the pharmaceutically acceptable salt thereof is from about 1 mg to about 200 mg per unit dose composition, about 5 mg to about 100 mg per unit dose composition, or about 25 mg to about 75 mg per unit dose composition.

Aspect 5. The unit dose composition of Aspect 1, wherein N-acetyl cysteine or the pharmaceutically acceptable salt thereof is from about 100 mg to about 1,000 mg per unit dose composition, about 250 mg to about 750 mg per unit dose composition, or about 400 mg to about 600 mg per unit dose composition.

Aspect 6. The unit dose composition of Aspect 1, wherein lutein or the pharmaceutically acceptable salt thereof is from about 0.1 mg to about 50 mg per unit dose composition, about 1 mg to about 25 mg per unit dose composition, or about 5 mg to about 20 mg per unit dose composition.

Aspect 7. The unit dose composition of Aspect 1, wherein taurine or the pharmaceutically acceptable salt thereof is from about 100 mg to about 2,000 mg per unit dose composition, about 500 mg to about 1,500 mg per unit dose composition, or about 750 mg to about 1,250 mg per unit dose composition.

Aspect 8. The unit dose composition of Aspect 1, wherein thiamine or the pharmaceutically acceptable salt thereof is from about 1 mg to about 200 mg per unit dose composition, about 5 mg to about 100 mg per unit dose composition, or about 10 mg to about 50 mg per unit dose composition.

Aspect 9. The unit dose composition of Aspect 1, wherein lycopene or the pharmaceutically acceptable salt thereof is from about 0.1 mg to about 50 mg per unit dose composition, about 1 mg to about 25 mg per unit dose composition, or about 5 mg to about 20 mg per unit dose composition.

Aspect 10. The unit dose composition of Aspect 1, wherein zeaxanthin or the pharmaceutically acceptable salt thereof is from about 0.1 mg to about 50 mg per unit dose composition, about 1 mg to about 25 mg per unit dose composition, or about 5 mg to about 20 mg per unit dose composition.

Aspect 11. The unit dose composition of Aspect 1, wherein the dose composition comprises
(a) N-acetyl carnosine or a pharmaceutically acceptable salt thereof in the amount of about 1 mg to about 100 mg per unit dose composition;
(b) S-acetyl glutathione or a pharmaceutically acceptable salt thereof in the amount of about 1 mg to about 200 mg per unit dose composition;
(c) pterostilbene or a pharmaceutically acceptable salt thereof in the amount of about 1 mg to about 200 mg per unit dose composition;
(d) N-acetyl cysteine or a pharmaceutically acceptable salt thereof in the amount of about 100 mg to about 1,000 mg per unit dose composition;
(e) lutein or a pharmaceutically acceptable salt thereof in the amount of about 0.1 mg to about 50 mg per unit dose composition;
(f) taurine or a pharmaceutically acceptable salt thereof in the amount of about 100 mg to about 2,000 mg per unit dose composition;
(g) thiamine or a pharmaceutically acceptable salt thereof in the amount of about 1 mg to about 200 mg per unit dose composition;
(h) lycopene or a pharmaceutically acceptable salt thereof in the amount of about 0.1 mg to about 50 mg per unit dose composition; and
(i) zeaxanthin or the pharmaceutically acceptable salt thereof is from about 0.1 mg to about 50 mg per unit dose composition.

Aspect 12. The unit dose composition of Aspect 1, wherein the dose composition comprises
(a) N-acetyl carnosine or a pharmaceutically acceptable salt thereof in the amount of about 10 mg to about 35 mg per unit dose composition;
(b) S-acetyl glutathione or a pharmaceutically acceptable salt thereof in the amount of about 25 mg to about 75 mg per unit dose composition;
(c) pterostilbene or a pharmaceutically acceptable salt thereof in the amount of about 25 mg to about 75 mg per unit dose composition;
(d) N-acetyl cysteine or a pharmaceutically acceptable salt thereof in the amount of about 400 mg to about 600 mg per unit dose composition;
(e) lutein or a pharmaceutically acceptable salt thereof in the amount of about 5 mg to about 20 mg per unit dose composition;
(f) taurine or a pharmaceutically acceptable salt thereof in the amount of about 750 mg to about 1,250 mg per unit dose composition;
(g) thiamine or a pharmaceutically acceptable salt thereof in the amount of about 10 mg to about 50 mg per unit dose composition;
(h) lycopene or a pharmaceutically acceptable salt thereof in the amount of about 5 mg to about 20 mg per unit dose composition; and
(i) zeaxanthin or the pharmaceutically acceptable salt thereof is from about 5 mg to about 20 mg per unit dose composition.

Aspect 13. The unit dose composition of Aspect 1, wherein the dose composition comprises
(a) N-acetyl carnosine or a pharmaceutically acceptable salt thereof in the amount of about 25 mg per unit dose composition;
(b) S-acetyl glutathione or a pharmaceutically acceptable salt thereof in the amount of about 50 mg per unit dose composition;
(c) pterostilbene or a pharmaceutically acceptable salt thereof in the amount of about 50 mg per unit dose composition;
(d) N-acetyl cysteine or a pharmaceutically acceptable salt thereof in the amount of about 500 mg per unit dose composition;
(e) lutein or a pharmaceutically acceptable salt thereof in the amount of about 10 mg per unit dose composition;
(f) taurine or a pharmaceutically acceptable salt thereof in the amount of about 900 mg per unit dose composition;
(g) thiamine or a pharmaceutically acceptable salt thereof in the amount of about 30 mg per unit dose composition;
(h) lycopene or a pharmaceutically acceptable salt thereof in the amount of about 10 mg per unit dose composition; and
(i) zeaxanthin or a pharmaceutically acceptable salt thereof in the amount of about 10 mg per unit dose composition.

Aspect 14. The unit dose composition of any one of Aspects 1-13, wherein the dose composition consists essentially of
(a) N-acetyl carnosine or a pharmaceutically acceptable salt thereof;
(b) S-acetyl glutathione or a pharmaceutically acceptable salt thereof;
(c) pterostilbene or a pharmaceutically acceptable salt thereof;
(d) N-acetyl cysteine or a pharmaceutically acceptable salt thereof;
(e) lutein or a pharmaceutically acceptable salt thereof;
(f) taurine or a pharmaceutically acceptable salt thereof;
(g) thiamine or a pharmaceutically acceptable salt thereof;
(h) lycopene or a pharmaceutically acceptable salt thereof; and
(i) zeaxanthin or a pharmaceutically acceptable salt thereof.

Aspect 15. The unit dose composition of any one of Aspects 1-13, wherein the dose composition consists of
(a) N-acetyl carnosine or a pharmaceutically acceptable salt thereof;
(b) S-acetyl glutathione or a pharmaceutically acceptable salt thereof;
(c) pterostilbene or a pharmaceutically acceptable salt thereof;
(d) N-acetyl cysteine or a pharmaceutically acceptable salt thereof;
(e) lutein or a pharmaceutically acceptable salt thereof;
(f) taurine or a pharmaceutically acceptable salt thereof;
(g) thiamine or a pharmaceutically acceptable salt thereof;
(h) lycopene or a pharmaceutically acceptable salt thereof; and
(i) zeaxanthin or a pharmaceutically acceptable salt thereof.

Aspect 16. The unit dose composition of any one of Aspects 1-15, wherein the pharmaceutically acceptable salt of (a)-(i) is sulfate salt, a chloride salt, or an aspartate salt.

Aspect 17. The unit dose composition in any one of Aspects 1-16, wherein the unit dose composition comprises a pharmaceutically acceptable carrier.

Aspect 18. The unit dose composition in any one of Aspects 1-16, wherein the unit dose composition does not include a pharmaceutically acceptable carrier.

Aspect 19. The unit dose composition in any one of Aspects 1-18, wherein the unit dose composition is a tablet or capsule.

Aspect 20. A pharmaceutical composition comprising the unit dose composition in any one of Aspects 1-16 and a pharmaceutically acceptable carrier.

Aspect 21. A method of improving eye function in a subject, the method comprising administering to the subject an effective amount of the unit dose composition in any one of Aspects 1-19.

Aspect 22. A method for treating or preventing eye disease in a subject, the method comprising administering to the subject an effective amount of the unit dose composition in any one of Aspects 1-19.

Aspect 23. The method of Aspects 21 or 22, wherein the subject has diabetes.

Aspect 24. The method of Aspects 21 or 22, wherein the subject is prediabetic.

Aspect 25. The method of any one of Aspects 21-24, wherein the unit dose composition reduces or prevents inflammation and oxidative stress on eye tissue.

Aspect 26. The method of any one of Aspects 21-24, wherein the unit dose composition reduces or prevents vision loss due to optic disc swelling, optic neuropathy, diabetic retinopathy, cataract, age-related macular degeneration (AMD), or glaucoma.

Aspect 27. The method of any one of Aspects 21-24, wherein the subject has optic disc swelling, optic neuropathy, diabetic retinopathy, cataract, age-related macular degeneration (AMD), or glaucoma.

Now having described the aspects of the present disclosure, in general, the following

Examples describe some additional aspects of the present disclosure. While aspects of the present disclosure are described in connection with the following examples and the corresponding text and figures, there is no intent to limit aspects of the present disclosure to this description. On the contrary, the intent is to cover all alternatives, modifications, and equivalents included within the spirit and scope of the present disclosure.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

1. A unit dose composition comprising
(a) N-acetyl carnosine or a pharmaceutically acceptable salt thereof;
(b) S-acetyl glutathione or a pharmaceutically acceptable salt thereof;
(c) pterostilbene or a pharmaceutically acceptable salt thereof;
(d) N-acetyl cysteine or a pharmaceutically acceptable salt thereof;
(e) lutein or a pharmaceutically acceptable salt thereof;
(f) taurine or a pharmaceutically acceptable salt thereof;
(g) thiamine or a pharmaceutically acceptable salt thereof;
(h) lycopene or a pharmaceutically acceptable salt thereof; and
(i) zeaxanthin or a pharmaceutically acceptable salt thereof.

2. The unit dose composition of claim 1, wherein N-acetyl carnosine or the pharmaceutically acceptable salt thereof is from about 1 mg to about 100 mg per unit dose composition, about 5 mg to about 50 mg per unit dose composition, or about 10 mg to about 35 mg per unit dose composition.

3. The unit dose composition of claim 1, wherein S-acetyl glutathione or the pharmaceutically acceptable salt thereof is from about 1 mg to about 200 mg per unit dose composition, about 5 mg to about 100 mg per unit dose composition, or about 25 mg to about 75 mg per unit dose composition.

4. The unit dose composition of claim 1, wherein pterostilbene or the pharmaceutically acceptable salt thereof is from about 1 mg to about 200 mg per unit dose composition, about 5 mg to about 100 mg per unit dose composition, or about 25 mg to about 75 mg per unit dose composition.

5. The unit dose composition of claim 1, wherein N-acetyl cysteine or the pharmaceutically acceptable salt thereof is from about 100 mg to about 1,000 mg per unit dose composition, about 250 mg to about 750 mg per unit dose composition, or about 400 mg to about 600 mg per unit dose composition.

6. The unit dose composition of claim 1, wherein lutein or the pharmaceutically acceptable salt thereof is from about 0.1 mg to about 50 mg per unit dose composition, about 1 mg to about 25 mg per unit dose composition, or about 5 mg to about 20 mg per unit dose composition.

7. The unit dose composition of claim 1, wherein taurine or the pharmaceutically acceptable salt thereof is from about 100 mg to about 2,000 mg per unit dose composition, about 500mg to about 1,500 mg per unit dose composition, or about 750 mg to about 1,250 mg per unit dose composition.

8. The unit dose composition of claim 1, wherein thiamine or the pharmaceutically acceptable salt thereof is from about 1 mg to about 200 mg per unit dose composition, about 5 mg to about 100 mg per unit dose composition, or about 10 mg to about 50 mg per unit dose composition.

9. The unit dose composition of claim 1, wherein lycopene or the pharmaceutically acceptable salt thereof is from about 0.1 mg to about 50 mg per unit dose composition, about 1 mg to about 25 mg per unit dose composition, or about 5 mg to about 20 mg per unit dose composition.

10. The unit dose composition of claim 1, wherein zeaxanthin or the pharmaceutically acceptable salt thereof is from about 0.1 mg to about 50 mg per unit dose composition, about 1 mg to about 25 mg per unit dose composition, or about 5 mg to about 20 mg per unit dose composition.

11. The unit dose composition of claim 1, wherein the dose composition comprises
(a) N-acetyl carnosine or a pharmaceutically acceptable salt thereof in the amount of about 1 mg to about 100 mg per unit dose composition;
(b) S-acetyl glutathione or a pharmaceutically acceptable salt thereof in the amount of about 1 mg to about 200 mg per unit dose composition;
(c) pterostilbene or a pharmaceutically acceptable salt thereof in the amount of about 1 mg to about 200 mg per unit dose composition;
(d) N-acetyl cysteine or a pharmaceutically acceptable salt thereof in the amount of about 100 mg to about 1,000 mg per unit dose composition;
(e) lutein or a pharmaceutically acceptable salt thereof in the amount of about 0.1 mg to about 50 mg per unit dose composition;
(f) taurine or a pharmaceutically acceptable salt thereof in the amount of about 100 mg to about 2,000 mg per unit dose composition;
(g) thiamine or a pharmaceutically acceptable salt thereof in the amount of about 1 mg to about 200 mg per unit dose composition;
(h) lycopene or a pharmaceutically acceptable salt thereof in the amount of about 0.1 mg to about 50 mg per unit dose composition; and
(i) zeaxanthin or the pharmaceutically acceptable salt thereof is from about 0.1 mg to about 50 mg per unit dose composition.

12. The unit dose composition of claim 1, wherein the dose composition comprises
(a) N-acetyl carnosine or a pharmaceutically acceptable salt thereof in the amount of about 10 mg to about 35 mg per unit dose composition;
(b) S-acetyl glutathione or a pharmaceutically acceptable salt thereof in the amount of about 25 mg to about 75 mg per unit dose composition;
(c) pterostilbene or a pharmaceutically acceptable salt thereof in the amount of about 25 mg to about 75 mg per unit dose composition;
(d) N-acetyl cysteine or a pharmaceutically acceptable salt thereof in the amount of about 400 mg to about 600 mg per unit dose composition;
(e) lutein or a pharmaceutically acceptable salt thereof in the amount of about 5 mg to about 20 mg per unit dose composition;
(f) taurine or a pharmaceutically acceptable salt thereof in the amount of about 750 mg to about 1,250 mg per unit dose composition;
(g) thiamine or a pharmaceutically acceptable salt thereof in the amount of about 10 mg to about 50 mg per unit dose composition;
(h) lycopene or a pharmaceutically acceptable salt thereof in the amount of about 5 mg to about 20 mg per unit dose composition; and
(i) zeaxanthin or the pharmaceutically acceptable salt thereof is from about 5 mg to about 20 mg per unit dose composition.

13. The unit dose composition of claim 1, wherein the dose composition comprises
(a) N-acetyl carnosine or a pharmaceutically acceptable salt thereof in the amount of about 25 mg per unit dose composition;
(b) S-acetyl glutathione or a pharmaceutically acceptable salt thereof in the amount of about 50 mg per unit dose composition;
(c) pterostilbene or a pharmaceutically acceptable salt thereof in the amount of about 50 mg per unit dose composition;
(d) N-acetyl cysteine or a pharmaceutically acceptable salt thereof in the amount of about 500 mg per unit dose composition;
(e) lutein or a pharmaceutically acceptable salt thereof in the amount of about 10 mg per unit dose composition;
(f) taurine or a pharmaceutically acceptable salt thereof in the amount of about 900 mg per unit dose composition;
(g) thiamine or a pharmaceutically acceptable salt thereof in the amount of about 30 mg per unit dose composition;
(h) lycopene or a pharmaceutically acceptable salt thereof in the amount of about 10 mg per unit dose composition; and
(i) zeaxanthin or a pharmaceutically acceptable salt thereof in the amount of about 10 mg per unit dose composition.

14. The unit dose composition of claim 1, wherein the dose composition consists essentially of
(a) N-acetyl carnosine or a pharmaceutically acceptable salt thereof;
(b) S-acetyl glutathione or a pharmaceutically acceptable salt thereof;
(c) pterostilbene or a pharmaceutically acceptable salt thereof;
(d) N-acetyl cysteine or a pharmaceutically acceptable salt thereof;
(e) lutein or a pharmaceutically acceptable salt thereof;
(f) taurine or a pharmaceutically acceptable salt thereof;
(g) thiamine or a pharmaceutically acceptable salt thereof;
(h) lycopene or a pharmaceutically acceptable salt thereof; and
(i) zeaxanthin or a pharmaceutically acceptable salt thereof.

15. The unit dose composition of claim 1, wherein the dose composition consists of
(a) N-acetyl carnosine or a pharmaceutically acceptable salt thereof;
(b) S-acetyl glutathione or a pharmaceutically acceptable salt thereof;
(c) pterostilbene or a pharmaceutically acceptable salt thereof;
(d) N-acetyl cysteine or a pharmaceutically acceptable salt thereof;
(e) lutein or a pharmaceutically acceptable salt thereof;
(f) taurine or a pharmaceutically acceptable salt thereof;
(g) thiamine or a pharmaceutically acceptable salt thereof;
(h) lycopene or a pharmaceutically acceptable salt thereof; and
(i) zeaxanthin or a pharmaceutically acceptable salt thereof.

16. The unit dose composition of claim 1, wherein the pharmaceutically acceptable salt of (a)-(i) is sulfate salt, a chloride salt, or an aspartate salt.

17. The unit dose composition of claim 1, wherein the unit dose composition comprises a pharmaceutically acceptable carrier.

18. The unit dose composition of claim 1, wherein the unit dose composition does not include a pharmaceutically acceptable carrier.

19. The unit dose composition of claim 1, wherein the unit dose composition is a tablet or capsule.

20. A pharmaceutical composition comprising the unit dose composition of claim 1 and a pharmaceutically acceptable carrier.

21. A method of improving eye function in a subject, the method comprising administering to the subject an effective amount of the unit dose composition of claim 1.

22. A method for treating or preventing eye disease in a subject, the method comprising administering to the subject an effective amount of the unit dose composition of claim 1.

23. The method of claim 21, wherein the subject has diabetes.

24. The method of claim 21, wherein the subject is prediabetic.

25. The method of claim 21, wherein the unit dose composition reduces or prevents inflammation and oxidative stress on eye tissue.

26. The method of claim 21, wherein the unit dose composition reduces or prevents vision loss due to optic disc swelling, optic neuropathy, diabetic retinopathy, cataract, age-related macular degeneration (AMD), or glaucoma.

27. The method of claim 21, wherein the subject has optic disc swelling, optic neuropathy, diabetic retinopathy, cataract, age-related macular degeneration (AMD), or glaucoma.
